(19) United States
(12) Patent Application Publication
(10) Pub. No.: US 2016/0263110 A1
(43) Pub. Date: Sep. 15, 2016

(54) USE OF MASITINIB FOR TREATMENT OF AMYOTROPHIC LATERAL SCLEROSIS

(71) Applicant: AB SCIENCE, Paris (FR)

(72) Inventors: Jean Pierre KINET, Lexington, MA (US); Alain MOUSSY, Paris (FR); Colin MANSFIELD, Ecully (FR)

(21) Appl. No.: 15/033,797

(22) PCT Filed: Nov. 4, 2014
(86) PCT No.: PCT/EP2014/073669

§ 371 Date: May 2, 2016

(30) Foreign Application Priority Data
Nov. 4, 2013 (EP) 13306513.6

(51) Int. Cl. A61K 31/496; A61K 31/428

(52) U.S. Cl. CPC A61K 31/496; A61K 31/428

(57) ABSTRACT

A treatment of patients afflicted with Amyotrophic Lateral Sclerosis (ALS), wherein the patients are treated with a tyrosine kinase inhibitor, mast cell inhibitor or c-Kit inhibitor, in particular masitinib, optionally in combination with at least one pharmaceutically active ingredient

The present invention relates to a method for treating patients afflicted with Amyotrophic Lateral Sclerosis (ALS), wherein said patients are treated with a tyrosine kinase inhibitor, mast cell inhibitor or c-Kit inhibitor, in particular masitinib, optionally in combination with at least one pharmaceutically active ingredient.

Clinical Features and Epidemiology of ALS

Amyotrophic lateral sclerosis is the most prevalent type of motor neuron disease. There is currently no effective disease-modifying treatment other than riluzole, which only has a modest effect on survival [Miller R. Riluzole for ALS: what is the evidence? Amyotroph. Lateral Scler. Other Motor Neuron Disord., 2003; 4: 135, 2] [Miller R G, et al. Riluzole for amyotrophic lateral sclerosis (ALS)/motor neuron disease (MND) Amyotroph. Lateral Scler. Other Motor Neuron Disord. 2003; 4: 191-206].

Amyotrophic lateral sclerosis is a rare degenerative disorder of large motor neurons of the cerebral cortex, brain stem and spinal cord that results in progressive wasting and paralysis of voluntary muscles. The incidence of ALS is currently approximately 2/100,000 per year and may be increasing. The lifetime ALS risk is 1 in 600 to 1 in 1000. Even though the incidence of ALS is similar to that of multiple sclerosis, the prevalence is only 4-6/100,000 (about 25,000 patients in the United States), due to the higher mortality rate. Fifty percent of ALS cases die within 3 years of onset of symptoms and 90% die within 5 years. The median age of onset is 55 years. The cause in most cases is unknown. Age and gender are the only risk factors repeatedly documented in epidemiological studies. There is a slight male predominance (3:2 male to female ratio) in sporadic ALS. The majority of ALS cases are sporadic (SALS); approximately 10% are familial (FALS). More than 100 point mutations in the gene encoding cytosolic copper-zinc superoxide dismutase (SOD1) have been demonstrated to cause typical FALS. Essential features of ALS are progressive signs and symptoms of lower motor neuron dysfunction (atrophy, cramps, and fasciculations) associated with corticospinal tract signs (spasticity, enhanced and pathological reflexes) in the absence of sensory findings.

Neuroinflammation is now established as an important aspect of pathology in ALS [McGeer P L, et al. Inflammatory processes in amyotrophic lateral sclerosis. Muscle Nerve. 2002; 26: 459-470] [Philips T, et al. Neuroinflammation in amyotrophic lateral sclerosis: role of glial activation in motor neuron disease. Lancet Neurol. 2011; 10: 253-263] [Weydt P, et al. Neuroinflammation in the pathogenesis of amyotrophic lateral sclerosis. Neuroreport. 2005; 16: 527-531]. Indeed, analysis of cerebrospinal fluid from ALS patients has shown dysregulation of a number of pro- and anti-inflammatory cytokines, and growth factors, including IL-6, IL-10 GM-CSF, VEGF and IFN-γ (6).

Overview of ALS Pathogenesis

Many causes of ALS have been proposed including toxicity from excess excitation of the motor neuron by transmitters such as glutamate, free radical-mediated oxidative cytotoxicity, neuroinflammation, mitochondrial dysfunction, autoimmune processes, cytoskeletal abnormalities, and aberrant activation of cyclo-oxygenase. It has also been suggested that atypical viral infections may trigger this disease (e.g. enteroviruses or atypical retroviruses). Whatever the cause, it is evident that there are multiple levels of cellular dysfunction as the disease progresses and that programmed cell death is activated in this disease. Mutations in the gene encoding SOD1 account for about 25% of cases of FALS or 2-3% of all ALS cases. Forced expression of high levels of a mutant SOD1 transgene causes progressive motor neuron disease in mice and rats. Additional genes implicated in ALS-like syndromes include ALS2, which codes for a guanine-nucleotide exchange-like factor and the dynactin gene. Five genetic defects have now been reported to cause FALS.

Current Therapies

The management of ALS is essentially symptoms-based.

No treatment prevents, halts, or reverses the disease, although riluzole use is associated with a slight prolongation of survival.

A summary of compounds tested in ALS are shown in the table below, grouped according to their hypothetical mechanisms of action.

List of drugs tested in ALS, with hypothetical mechanisms of action and
currently available results

| Mechanism of Action | Mouse model | Human Safety | Benefits |
|---|---|---|---|
| Antiglutamatergic | Overall positive | Relatively safe | Positive transient impact |
| | effects | | on survival with Riluzole, |
| | | | no benefit with other |
| | | | compounds |
| Antioxidant | Overall positive | Relatively safe | No benefit on survival |
| | effects | | |
| Antiapoptotic | Overall positive | Relatively safe | No benefit on survival |
| | effects | | |
| Anti-aggregation | Discordant | Relatively safe | No efficacy data |
| | results | | |
| Neuroprotective | Overall positive | Relatively safe | No benefit on survival |
| | effects | | |
| Anti-Inflammatory: | | | |
| Other than PKI* | Overall positive | Relatively safe | Limited data. Studies |
| | effects | | underway. Trend for |
| PKI** | Overall positive | Relatively safe | survival benefit |
| | effects | | |

*PKI: Protein kinase inhibitor;
**Tamoxifen
Source: Zoccolella et al. Current and emerging treatments for amyotrophic lateral sclerosis. Neuropsychiatric Disease and Treatment 2009: 5 577-595.

There remains a high unmet medical need for effective drugs in the treatment of ALS.

### AIMS OF THE INVENTION

The invention aims to solve the technical problem of providing an active ingredient for the treatment of ALS.

The invention also aims to solve the technical problem of providing an active ingredient for an efficient treatment of ALS, especially in human patients.

The invention also aims to solve the technical problem of providing an active ingredient that improves prior art methods for the treatment of ALS.

The invention aims to provide an efficient treatment for ALS at an appropriate dose, route of administration, and daily intake.

### SUMMARY OF THE INVENTION

In Amyotrophic Lateral Sclerosis (ALS), both the upper motor-neurons (located in the brain) and the lower motor-neurons (located in the spinal cord) degenerate or die, ceasing to send messages to muscles. A common pathological hallmark in ALS is the presence of ubiquitin-immunoreactive cytoplasmic inclusions in degenerating neurons, followed by a strong inflammatory reaction. Evidence suggests that neuroinflammation is a pathological characteristic of ALS and could therefore represent a potential therapeutic target for a pharmacological agent to help treat this severe disease [Weydt P, et al. Neuro-inflammation as a therapeutic target in amyotrophic lateral sclerosis. Curr Opin Investig Drugs. 2002 December; 3(12):1720-4.; and references therein]. Indeed, this analysis has since been corroborated by studies that have observed inflammatory markers in affected neural tissues of ALS patients, suggesting that inflammation in ALS spinal cord and cortex is based on innate immune responses by macrophages including mast cells. It has also been shown that mast cells infiltrate the spinal cord of ALS patients [Graves M C, et al. Inflammation in amyotrophic lateral sclerosis spinal cord and brain is mediated by activated macrophages, mast cells and T cells. Amyotroph Lateral Scler Other Motor Neuron Disord. 2004 December; 5(4):213-9]. In addition, elevated TNF alpha levels, which are expressed through mast cells, have been reported in ALS patients and have been shown to induce motor-neuron death [Sayed et al. Meningeal mast cells affect early T cell central nervous system infiltration and blood-brain barrier integrity through TNF: a role for neutrophil recruitment?.J Immunol. 2010 Jun. 15; 184(12):6891-900].

Mast cells, which are found on both sides of the blood-brain barrier (BBB), play an important role in sustaining the inflammatory network [Theoharides T C. Mast cells and stress—a psychoneuroimmunological perspective. J Clin Psychopharmacol. 2002 April; 22(2):103-8] [Stassen M, et al. Mast cells and inflammation. Arch Immunol Ther Exp (Warsz). 2002; 50(3):179-85] [Kinet J P. The essential role of mast cells in orchestrating inflammation. Immunol Rev. 2007 Jun;217:5-7]. Moreover, it has been shown that mast cells are able to cross the BBB and their numbers may rapidly increase in response to physiological manipulations [Nautiyal K, et al. Brain mast cells link the immune system to anxiety-like behavior. Proc Natl Acad Sci USA. 2008 November; 18; 105(46): 18053-18057] [Theoharides T C, et al. Critical role of mast cells in inflammatory diseases and the effect of acute stress. J NeuroimmunoL 2004 January; 146(1-2):1-12] [Silverman A J, et al. Mast cells migrate from blood to brain. J Neurosci 2000, 20:401-408]. Hence, mast cells may actively participate in the pathogenesis of ALS, in part because they release large amounts of proinflammatory mediators that sustain the inflammatory network of the central nervous system.

Additionally, the role of BBB (Brain-Blood Barrier) dysfunction and blood-spinal cord barrier (BSCB) dysfunction in ALS has implications for disease pathogenesis. The first indication that human BBB might be affected in ALS came in 1984 when abnormal IgG and albumin levels were observed in patients' cerebrospinal fluid [Leonardi A, et al. Cerebrospinal fluid (CSF) findings in amyotrophic lateral sclerosis. J Neurol. 1984; 231(2):75-8]. More recently it has been shown that disruption of the BBB and BSCB occur in areas of motor neuron degeneration in the brain and spinal cord of a G93A SOD1 mice modeling ALS at both early and late stages of disease [Garbuzova-Davis S, et al. Evidence of compromised blood-spinal cord barrier in early and late symptomatic SOD 1 mice modeling ALS. PLoS One. 2007 Nov. 21; 2(11):e1205]. Perivascular localized mast cells secrete numerous vasoactive molecules that regulate BBB permeability [Secor V H, et al. Mast cells are essential for early onset and severe disease in a murine model of multiple sclerosis. J Exp Med. 2000 Mar. 6; 191(5):813-22] [Esposito P, et al. Corticotropin-releasing hormone and brain mast cells regulate blood-brain-barrier permeability induced by acute stress. J Pharmacol Exp Ther. 2002; 303:1061-1066] [Esposito P, et al. Acute stress increases permeability of the blood-brain-barrier through activation of brain mast cells. Brain Res. 2001 January 5; 888(1):117-127] [Zhuang X, et al. Brain mast cell degranulation regulates blood-brain barrier. J Neurobiol. 1996 December; 31(4):393-403]. Inhibition of mast cell mediators and apoptosis of mast cells localized at the BBB would effectively reduce BBB permeability, thereby reinforcing its integrity and stemming the accumulation of exogenous damaging factors in the brain.

A motor neuron-specific death pathway has been suggested for ALS based on the finding that motor neurons isolated from transgenic SOD1 mutant mice were more sensitive to Fas- or NO-triggered cell death than wild-type motor neurons [Raoul C, et al. Motoneuron death triggered by a specific pathway downstream of Fas. potentiation by ALS-linked SOD1 mutations. Neuron. 2002 Sep. 12; 35(6):1067-83]. In vitro experiments showed that mast cell activation lead to neuronal damage by astrocyte/NO-dependent and -independent pathways [Skaper S D, et al. Mast cell activation causes delayed neurodegeneration in mixed hippocampal cultures via the nitric oxide pathway. J Neurochem. 1996; 66:1157-1166]. Specifically, the cognate mast cell line RBL-2H3, when subjected to an antigenic stimulus, released TNF-a which, together with exogenous interleukin-1β (or interferon-γ), induced astroglia to produce neurotoxic quantities of NO. It has also been reported that mast cells can be a source of NO derivatives, which they synthesize spontaneously or following activation, depending on their subtype [Bidri M, et al. Mast cells as a source and target for nitric oxide. Int Immunopharmacol. 2001; 1:1543-1558]. This evidence supports the notion that mast cells, which can be found in close vicinity to neurons, could influence the survival and functions of NO-sensitive cells and through this mechanism participate in the pathophysiology of chronic neurodegenerative diseases of the nervous system. It further suggests that down-modulation of mast cell activation in such conditions could be of therapeutic benefit.

Hence, mast cells appear to play a central role in the pathogenesis of ALS, in part through their contributions to neuroinflammation, NO mediated neuronal death, and effect on the BBB integrity.

The present invention relates to a method for the treatment of ALS wherein said method comprises administering to a mammal in need thereof, at least one tyrosine kinase inhibitor, mast cell inhibitor or c-Kit inhibitor.

In one embodiment, tyrosine kinase inhibitor, mast cell inhibitor or c-Kit inhibitor is administered to a human patient.

In one embodiment, said tyrosine kinase inhibitor, mast cell inhibitor or c-Kit inhibitor is administered in combination with at least one pharmaceutically active ingredient. Said pharmaceutically active ingredient is preferably active in the treatment of ALS. Such pharmaceutically active ingredient is preferably an antiglutamate compound, especially riluzole (6-(trifluoromethoxy)benzothiazol-2-amine); topiramate (2,3:4,5-Bis-O-(1-methylethylidene)-beta-D-fructopyranose sulfamate); gabapentin (2-[1-(aminomethyl)cyclohexyl]acetic acid); lamotrigine (6-(2,3-dichlorophenyl)-1,2,4-triazine-3,5-diamine); talampanel ((8R)-7-Acetyl-5-(4-aminophenyl)-8,9-dihydro-8-methyl-7H-1,3-dioxolo[4,5-h] [2,3]benzodiazepine); ceftriaxone ((6R,7R)-7-[[(2Z)-2-(2-amino-1,3-thiazol-4-yl)-2-methoxyiminoacetyl]amino]-3-[(2-methyl-5,6-dioxo-1H-1,2,4-triazin-3-yl)sulfanylmethyl]-8-oxo-5-thia-1-azabicyclo[4.2.0]oct-2-ene-2-carboxylic acid); an inhibitor of Glutamate carboxypeptidase II, preferably said other pharmaceutically active ingredient is riluzole.

Preferably, said tyrosine kinase inhibitor or a mast cell inhibitor is an inhibitor of kinase activity selected from the tyrosine kinases of: c-Kit, PDGFR, Lyn and Fyn.

Preferably, said tyrosine kinase inhibitor, mast cell inhibitor or c-Kit inhibitor is masitinib or a pharmaceutically acceptable salt thereof, and even more preferably, a masitinib mesilate salt.

In one embodiment, said tyrosine kinase inhibitor, mast cell inhibitor or c-Kit inhibitor, preferably masitinib is administered at a daily dose of 1.0 to 12.0 mg/kg/day (mg per kg bodyweight per day).

In one embodiment, said tyrosine kinase inhibitor, mast cell inhibitor or c-Kit inhibitor, preferably masitinib is administered at a dose of 2.5 to 9.5 mg/kg/day.

In one embodiment, said tyrosine kinase inhibitor, mast cell inhibitor or c-Kit inhibitor, preferably masitinib is administered at a dose of 3.0 to 9.0 mg/kg/day.

In one embodiment, said tyrosine kinase inhibitor, mast cell inhibitor or c-Kit inhibitor, preferably masitinib is administered at a dose of 1.5, 3.0, 4.5, 6.0, 7.5, or 9.0 mg/kg/day.

In one embodiment, said tyrosine kinase inhibitor, mast cell inhibitor or c-Kit inhibitor, preferably masitinib is administered at a starting dose of 4.0 to 10.0 mg/kg/day.

In one embodiment, said tyrosine kinase inhibitor, mast cell inhibitor or c-Kit inhibitor, preferably masitinib is administered at a starting dose of 2.5 to 5.5 mg/kg/day.

In one embodiment, said tyrosine kinase inhibitor, mast cell inhibitor or c-Kit inhibitor, preferably masitinib is dose escalated by increments of 1.5 mg/kg/day to reach a maximum of 9.0 mg/kg/day.

In one embodiment, said tyrosine kinase inhibitor, mast cell inhibitor or c-Kit inhibitor, preferably masitinib is dose reduced by increments of 1.5 mg/kg/day to reach a minimum of 1.5 mg/kg/day.

Any dose indicated herein refers to the amount of active ingredient as such, not to its salt form.

Safety data also suggest that masitinib is well-tolerated at a dosage up to 6 mg/kg/day in non-oncology indications with no obvious difference between 3 mg/kg/day and 6 mg/kg/day.

Given that the masitinib dose in mg/kg/day used in the described dose regimens refers to the amount of active ingredient masitinib, compositional variations of a pharmaceutically acceptable salt of masitinib mesilate will not change the said dose regimens.

In one embodiment, said tyrosine kinase inhibitor, mast cell inhibitor or c-Kit inhibitor, preferably masitinib is administered orally.

In one embodiment, said tyrosine kinase inhibitor, mast cell inhibitor or c-Kit inhibitor, preferably masitinib inhibitor is administered once or twice a day.

In one embodiment, said other pharmaceutically active ingredient is riluzole. Riluzole may be administered at a dose of 50 to 200 mg/day, for example 50, 100, or 200 mg/day, preferably 50 mg twice daily.

The invention specifically relates to the combination of masitinib and riluzole.

In one embodiment, said tyrosine kinase inhibitor, mast cell inhibitor or c-Kit inhibitor is administered in combination with said at least one pharmaceutically active ingredient in a combined preparation for simultaneous, separate, or sequential use.

The invention also relates to a tyrosine kinase inhibitor, mast cell inhibitor or c-Kit inhibitor, preferably masitinib, as defined according to the present invention, for use in a treatment of ALS.

The invention also relates to a tyrosine kinase tyrosine kinase inhibitor, mast cell inhibitor or c-Kit inhibitor, preferably masitinib, as defined according to the present invention, for use in a treatment of ALS, in combination with at least pharmaceutically active ingredient, preferably an antiglutamate compound, especially riluzole; topiramate; gabapentin; lamotrigine; talampanel; ceftriaxone; an inhibitor of Glutamate carboxypeptidase II, preferably said other pharmaceutically active ingredient is riluzole.

The invention also relates to a pharmaceutical composition or kit comprising a tyrosine kinase inhibitor, mast cell inhibitor or c-Kit inhibitor, preferably masitinib, for use in a method for the treatment of ALS as defined according to the present invention,.

The invention also relates to a pharmaceutical composition or kit comprising a tyrosine kinase inhibitor, mast cell inhibitor or c-Kit inhibitor, preferably masitinib, and at least one other pharmaceutically active ingredient, preferably an antiglutamate compound, especially riluzole; topiramate; gabapentin; lamotrigine; talampanel; ceftriaxone ; an inhibitor of Glutamate carboxypeptidase II, preferably said other pharmaceutically active ingredient is riluzole.

The invention also relates to the use of a tyrosine kinase inhibitor, mast cell inhibitor or c-Kit inhibitor, preferably masitinib, for the preparation of a medicament, or a pharmaceutical composition, for the treatment of ALS, optionally in combination with at least one other pharmaceutically active ingredient, preferably an antiglutamate compound, especially riluzole; topiramate; gabapentin; lamotrigine; talampanel; ceftriaxone ; an inhibitor of Glutamate carboxypeptidase II, preferably said other pharmaceutically active ingredient is riluzole, as defined according to the invention.

The terms “as defined according to the invention” refer to any embodiments or aspects of the invention alone or in combination without limitation, including any preferred embodiments and variants, including any embodiments and features relating to tyrosine kinase inhibitor, mast cell inhibitor or c-Kit inhibitor, preferably masitinib, the method of treatment of ALS, pharmaceutical compositions and any combination with other pharmaceutically active ingredient(s), preferably an antiglutamate compound, especially riluzole; topiramate; gabapentin; lamotrigine; talampanel; ceftriaxone ; an inhibitor of Glutamate carboxypeptidase II, preferably said other pharmaceutically active ingredient is riluzole. “Masitinib “designates also an acceptable salt thereof, especially masitinib mesilate, even not explicitly stated.

The tyrosine kinase inhibitor, mast cell inhibitor or c-Kit inhibitor, and the optional at least one pharmaceutically active ingredient, are administered in a dosage regimen that comprises a therapeutically effective amount.

### DESCRIPTION OF THE INVENTION

Tyrosine kinases are receptor type or non-receptor type proteins, which transfer the terminal phosphate of ATP to tyrosine residues of proteins thereby activating or inactivating signal transduction pathways. These proteins are known to be involved in many cellular mechanisms, which in case of disruption, lead to disorders such as abnormal cell proliferation and migration as well as inflammation. A tyrosine kinase inhibitor is a drug that inhibits tyrosine kinases, thereby interfering with signaling processes within cells. Blocking such processes can stop the cell growing and dividing.

In one embodiment, the tyrosine kinase inhibitor of the invention has the following formula [A]:

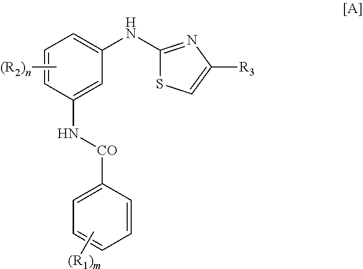

wherein R1 and R2, are selected independently from hydrogen, halogen, a linear or branched alkyl, cycloalkyl group containing from 1 to 10 carbon atoms, trifluoromethyl, alkoxy, cyano, dialkylamino, and a solubilizing group,

m is 0-5 and n is 0-4;

the group R3 is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, cyano and alkoxy;

(ii) a heteroaryl group such as 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy;

or a pharmaceutically acceptable salt or solvate thereof.

Tyrosine kinase inhibitors of formula [A] can preferably be used as c-Kit inhibitors.

Unless otherwise specified, the below terms used herein are defined as follows:

As used herein, the term an “aryl group” means a monocyclic or polycyclic-aromatic radical comprising carbon and hydrogen atoms. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more substituents. In one embodiment, the aryl group is a monocyclic ring, wherein the ring comprises 6 carbon atoms, referred to herein as “(C6)aryl”.

As used herein, the term “alkyl group” means a saturated straight chain or branched non-cyclic hydrocarbon having from 1 to 10 carbon atoms. Representative saturated straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. Alkyl groups included in compounds of this invention may be optionally substituted with one or more substituents.

As used herein, the term “alkoxy” refers to an alkyl group which is attached to another moiety by an oxygen atom. Examples of alkoxy groups include methoxy, isopropoxy, ethoxy, tert-butoxy, and the like. Alkoxy groups may be optionally substituted with one or more substituents.

As used herein, the term “heteroaryl” or like terms means a monocyclic or polycyclic heteroaromatic ring comprising carbon atom ring members and one or more heteroatom ring members (such as, for example, oxygen, sulfur or nitrogen). Typically, a heteroaryl group has from 1 to about 5 heteroatom ring members and from 1 to about 14 carbon atom ring members. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzo(b)thienyl. A heteroatom may be substituted with a protecting group known to those of ordinary skill in the art, for example, the hydrogen on a nitrogen may be substituted with a tert-butoxycarbonyl group. Heteroaryl groups may be optionally substituted with one or more substituents. In addition, nitrogen or sulfur heteroatom ring members may be oxidized. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings.

The term “heterocycle” as used herein, refers collectively to heterocycloalkyl groups and heteroaryl groups.

As used herein, the term “heterocycloalkyl” means a monocyclic or polycyclic group having at least one heteroatom selected from O, N or S, and which has 2-11 carbon atoms, which may be saturated or unsaturated, but is not aromatic. Examples of heterocycloalkyl groups include (but are not limited to): piperidinyl, piperazinyl, 2-oxopiperazinyl, 2-oxopiperidinyl, 2-oxopyrrolidinyl, 4-piperidonyl, pyrrolidinyl, hydantoinyl, valerolactamyl, oxiranyl, oxetanyl, tetrahydropyranyl, tetrahydrothiopyranyl, tetrahydropyrindinyl, tetrahydropyrimidinyl, tetrahydrothiopyranyl sulfone, tetrahydrothiopyranyl sulfoxide, morpholinyl, thiomorpholinyl, thiomorpholinyl sulfoxide, thiomorpholinyl sulfone, 1,3-dioxolane, tetrahydrofuranyl, dihydrofuranyl-2-one, tetrahydrothienyl, and tetrahydro-1,1-dioxothienyl. Typically, monocyclic heterocycloalkyl groups have 3 to 7 members. Preferred 3 to 7 membered monocyclic heterocycloalkyl groups are those having 5 or 6 ring atoms. A heteroatom may be substituted with a protecting group known to those of ordinary skill in the art, for example, the hydrogen on a nitrogen may be substituted with a tert-butoxycarbonyl group. Furthermore, heterocycloalkyl groups may be optionally substituted with one or more substituents. In addition, the point of attachment of a heterocyclic ring to another group may be at either a carbon atom or a heteroatom of a heterocyclic ring. Only stable isomers of such substituted heterocyclic groups are contemplated in this definition.

As used herein the term “substituent” or “substituted” means that a hydrogen radical on a compound or group is replaced with any desired group that is substantially stable to reaction conditions in an unprotected form or when protected using a protecting group. Examples of preferred substituents are those found in the exemplary compounds and embodiments disclosed herein, as well as halogen (chloro, iodo, bromo, or fluoro); alkyl; alkenyl; alkynyl; hydroxy; alkoxy; nitro; thiol; thioether; imine; cyano; amido; phosphonato; phosphine; carboxyl; thiocarbonyl; sulfonyl; sulfonamide; ketone; aldehyde; ester; oxygen (—O); haloalkyl (e.g., trifluoromethyl); cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl), monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); CO2CH3; CONH2; OCH2CONH2; NH2; SO2NH2; OCHF2; CF3; OCF3; and such moieties may also be optionally substituted by a fused-ring structure or bridge, for example —OCH2O—. These substituents may optionally be further substituted with a substituent selected from such groups. In certain embodiments, the term “substituent” or the adjective “substituted” refers to a substituent selected from the group consisting of an alkyl, an alkenyl, an alkynyl, an cycloalkyl, an cycloalkenyl, a heterocycloalkyl, an aryl, a heteroaryl, an aralkyl, a heteraralkyl, a haloalkyl, —C(O)NR11R12, —NR13C(O)R14, a halo, —OR13, cyano, nitro, a haloalkoxy, —C(O)R13, —NR11R12, —SR13, —C(O)OR13, —OC(0)R13, —NR13C(O)NR11R12, —OC(O)NR11R12, —NR13C(O)OR14, —S(O)rR13, —NR13S(O)rR14,—OS(O)rR14, S(OrNR11R12, —O, —S, and —N—R13, wherein r is 1 or 2; R11 and R12, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R11 and R12 taken together with the nitrogen to which they are attached is optionally substituted heterocycloalkyl or optionally substituted heteroaryl; and R13 and R14 for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl.

In certain embodiments, the term “substituent” or the adjective “substituted” refers to a solubilizing group.

The term “solubilizing group” means any group which can be substantially ionized and that enables the compound to be soluble in a desired solvent, such as, for example, water or water-containing solvent. Furthermore, the solubilizing group can be one that increases the compound or complex's lipophilicity. Typically, the solubilizing group is selected from alkyl group substituted with one or more heteroatoms such as N, 0, S, each optionally substituted with alkyl group substituted independently with alkoxy, amino, alkylamino, dialkylamino, carboxyl, cyano, or substituted with cycloheteroalkyl or heteroaryl, or a phosphate, or a sulfate, or a carboxylic acid. For example, by “solubilizing group” it is referred herein to one of the following:
an alkyl, cycloalkyl, aryl, heretoaryl group comprising either at least one nitrogen or oxygen heteroatom or which group is substituted by at least one amino group or oxo group;
an amino group which may be a saturated cyclic amino group which may be substituted by a group consisting of alkyl, alkoxycarbonyl, halogen, haloalkyl, hydroxyalkyl, amino, monoalkylamino, dialkylamino, carbamoyl, monoalkylcarbamoyl and dialkylcarbamoyl;
one of the structures a) to i) shown below, wherein the wavy line and the arrow line correspond to the point of attachment to core structure of Formula [A]:

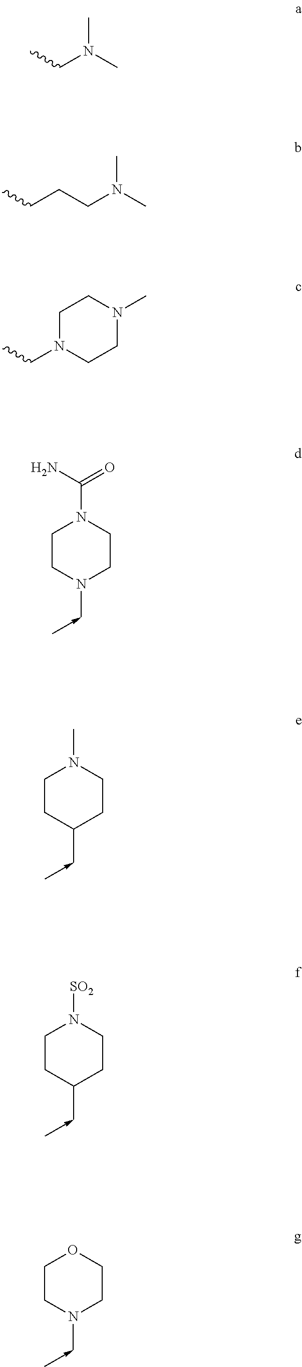

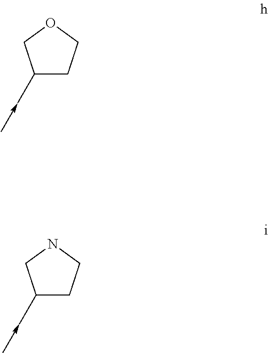

The term “cycloalkyl” means a saturated cyclic alkyl radical having from 3 to 10 carbon atoms. Representative cycloalkyls include cyclopropyl, 1-methylcyclopropyl, cyclobutyl, cyclopentyl, cyclohexyl, cycloheptyl, cyclooctyl, cyclononyl, and cyclodecyl. Cycloalkyl groups can be optionally substituted with one or more substituents.

The term “halogen” means -F, -CI, -Br or -I.

In a particular embodiment the tyrosine kinase inhibitor of the invention has general formula [B],

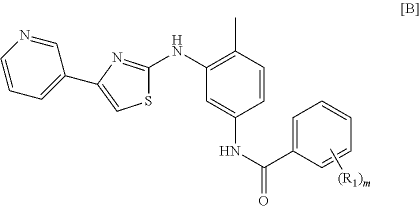

wherein:

R1 is selected independently from hydrogen, halogen, a linear or branched alkyl, cycloalkyl group containing from 1 to 10 carbon atoms, trifluoromethyl, alkoxy, amino, alkylamino, dialkylamino, solubilizing group, and m is 0-5.

In one embodiment, the tyrosine kinase inhibitor, mast cell inhibitor or c-Kit inhibitor is masitinib or a pharmaceutically acceptable salt thereof, more preferably masitinib mesilate.

Masitinib is a c-Kit / PDGFR inhibitor with a potent anti mast cell action.

New potent and selective c-Kit, platelet derived growth factor receptor (PDGFR) inhibitors are 2-(3-aminoaryl)amino-4-aryl-thiazoles described in AB Science's PCT application WO 2004/014903.

Masitinib is a small molecule drug, selectively inhibiting specific tyrosine kinases such as c-Kit, PDGFR, Lyn, and Fyn without inhibiting, at therapeutic doses, kinases associated with known toxicities (i.e. those tyrosine kinases or tyrosine kinase receptors attributed to possible tyrosine kinase inhibitor cardiac toxicity, including ABL, KDR and Src) [Dubreuil et al., 2009, PLoS ONE 2009.4(9):e7258] [Davis et al., Nat Biotechnol 2011, 29(11): 1046-51]. The chemical name for masitinib is 4-(4-methylpiperazin-1-ylmethyl)-N-[4-methyl-3-(4-pyridin-3yl thiazol-2-ylamino) phenyl]benzamide—CAS number 790299-79-5, and the structure is shown below. Masitinib was first described in U.S. Pat. No. 7,423,055 and EP1525200B1. A detailed procedure for the synthesis of masitinib mesilate is given in WO2008/098949.

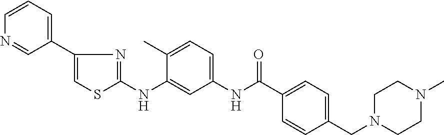 [SMILES: Cc1ccc(NC(=O)c2ccc(CN3CCN(C)CC3)cc2)cc1Nc1nc(-c2cccnc2)cs1]

Masitinib's main kinase target is c-Kit, for which it has been shown to exert a strong inhibitory effect on wild-type and juxtamembrane-mutated c-Kit receptors, resulting in cell cycle arrest and apoptosis of cell lines dependent on c-Kit signaling [Dubreuil et al., 2009, PLoS ONE, 4(9):e7258]. In vitro, masitinib demonstrated high activity and selectivity against c-Kit, inhibiting recombinant human wild-type c-Kit with an half inhibitory concentration (1050) of 200±40 nM and blocking stem cell factor-induced proliferation and c-Kit tyrosine phosphorylation with an 1050 of 150±80 nM in Ba/F3 cells expressing human or mouse wild-type c-Kit. In addition to its anti-proliferative properties, masitinib can also regulate the activation of mast cells through its targeting of Lyn and Fyn, key components of the transduction pathway leading to IgE induced degranulation [Gilfillan et al., 2006, Nat Rev Immunol, 6:218-230] [Gilfillan et al., 2009, Immunological Reviews, 228:149-169]. This can be observed in the inhibition of FccRI-mediated degranulation of human cord blood mast cells [Dubreuil et al., 2009, PLoS ONE;4(9):e7258]. Masitinib is also an inhibitor of PDGFR α and β receptors. Recombinant assays show that masitinib inhibits the in vitro protein kinase activity of PDGFR-α and β with 1050 values of 540±60 nM and 800±120 nM. In Ba/F3 cells expressing PDGFR-α, masitinib inhibited PDGF-BB-stimulated proliferation and PDGFR-α tyrosine phosphorylation with an 1050 of 300±5 /nM.

The present invention relates to a method for the treatment of ALS in a mammal, and especially a human patient, wherein said method comprises administering to a human patient in need thereof, a tyrosine kinase inhibitor, mast cell inhibitor or c-Kit inhibitor, especially masitinib or a pharmaceutically acceptable salt thereof, optionally combined with at least one pharmaceutically active ingredient.

In relation to the present invention, the term “treatment” (and its various grammatical forms) refers to preventing, curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessary all symptoms) of a disease or attenuating the progression of a disease.

Advantageously, the use or method comprises a long term administration of an effective amount of said tyrosine kinase inhibitor, mast cell inhibitor or c-Kit inhibitor, especially masitinib or a pharmaceutically acceptable salt thereof, over more than 3 months, preferably more than 6 months.

As is known to the person skilled in the art, various forms of excipients can be used adapted to the mode of administration and some of them can promote the effectiveness of the active molecule, e.g. by promoting a release profile rendering this active molecule overall more effective for the treatment desired.

The pharmaceutical compositions of the invention are thus able to be administered in various forms, more specially for example in an injectable, pulverizable or ingestible form, for example via the intramuscular, intravenous, subcutaneous, intradermal, oral, topical, rectal, vaginal, ophthalmic, nasal, transdermal or parenteral route. A preferred route is oral administration. The present invention notably covers the use of a compound according to the present invention for the manufacture of pharmaceutical composition.

Such medicament can take the form of a pharmaceutical composition adapted for oral administration, which can be formulated using pharmaceutically acceptable carriers well known in the art in suitable dosages. Such carriers enable the pharmaceutical compositions to be formulated as tablets, pills, dragees, capsules, liquids, gels, syrups, slurries, suspensions, and the like, for ingestion by the patient. In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

According to a particular embodiment, the composition of the invention is an oral composition.

In one embodiment, compositions according to the invention may be in the form of tablets.

In one embodiment, composition according to the invention may comprise from 50 to 500 mg of said tyrosine kinase inhibitor, mast cell inhibitor or c-Kit inhibitor, especially masitinib or a pharmaceutically acceptable salt thereof. More particularly, the composition may comprise from 100 to 500 mg of said tyrosine kinase inhibitor, mast cell inhibitor or c-Kit inhibitor, especially masitinib or a pharmaceutically acceptable salt thereof, for example, 100, 200, 300, 400, or 500 mg.

In the drawings:

FIG. 1 is the Kaplan-Meier plot of survival analysis according to Example 1.

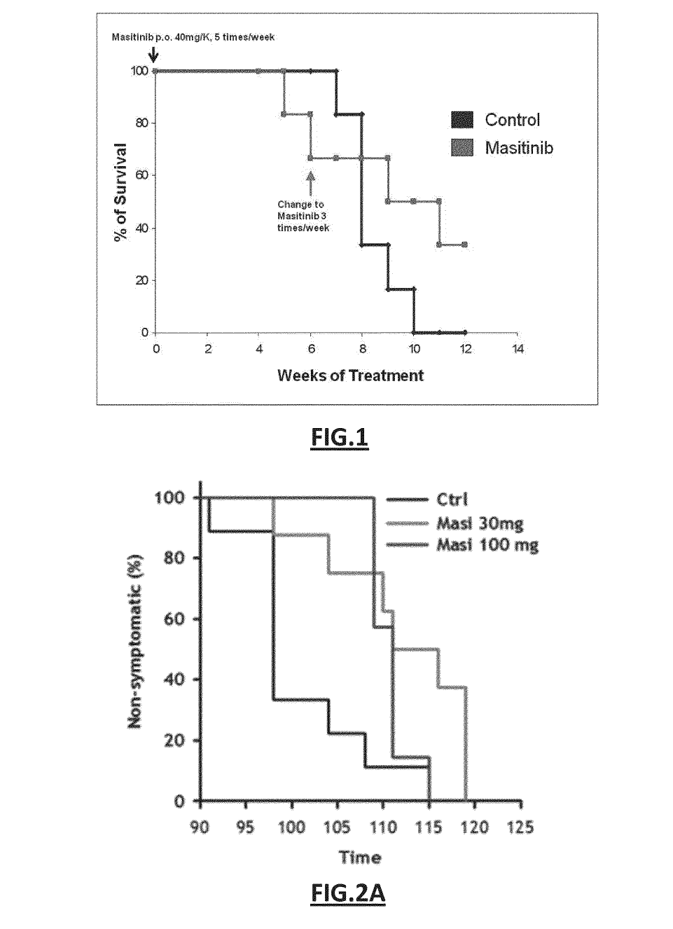

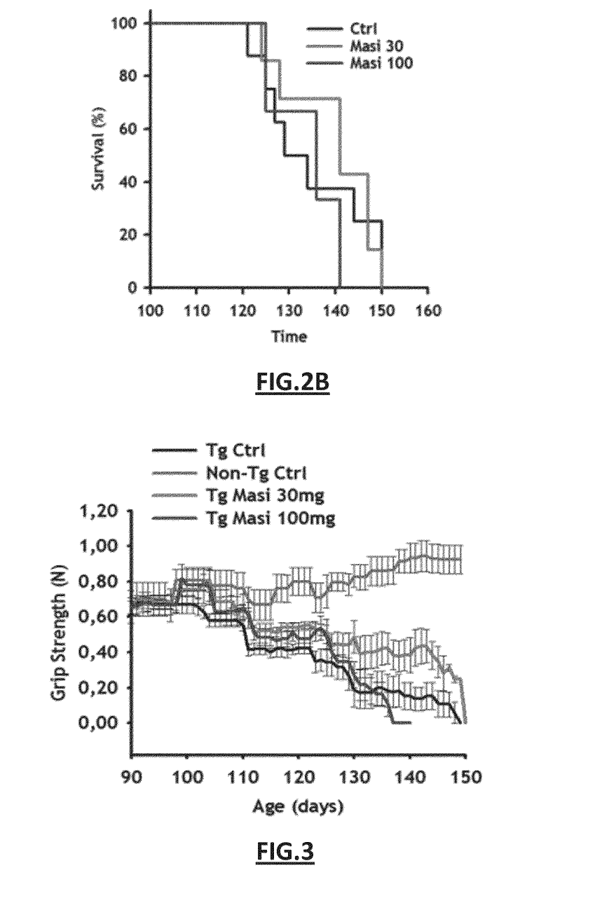

FIG. 2 shows Kaplan-Meyer symptoms onset (left -2A) and survival (right −2B) curves from B6SJL-Tg SOD1 G93A mice treated with masitinib at 30 mg/kg/day (Masi 30), masitinib at 100 (Masi 100) mg/kg/day, or water (Ctrl) as indicated.

FIG. 3 shows hind-limb grip strength for B6SJL-Tg SODG93A mice treated with masitinib at 30 or 100 mg/kg/day, B6SJL-Tg SODG93A mice treated with water (Tg Ctrl, positive control), and Non-Tg SODG93A mice (Non-Tg Ctrl, negative control).

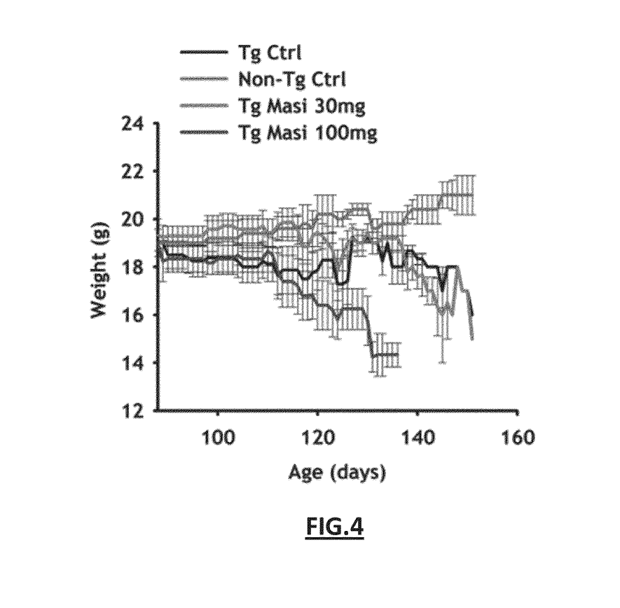

FIG. 4 shows weight records for B6SJL-Tg SODG93A mice treated with masitinib at 30 or 100 mg/kg/day, B6SJL-Tg SODG93A mice treated with water (Tg Ctrl, positive control), and Non-Tg SODG93A mice (Non-Tg Cntl, negative control).

The present invention is further illustrated by means of the following examples.

The data presented in these examples, and also in parts of the patent Description, are in part taken from preliminary analysis and as such represent a close approximation to the final, validated dataset.

### EXAMPLES

### Example 1

### Effect of Masitinib on Amylotrophic Lateral Sclerosis in SOD1G93A Rats

To investigate the hypothesis that masitinib's targeted inhibitory action may reduce the symptoms of ALS, its efficacy was assessed in rats expressing human Cu—Zn superoxide dismutase (SOD1) mutations. These rats develop a motor syndrome with symptoms and pathological features of the human disease [Howland D S, et al. Focal loss of the glutamate transporter EAAT2 in a transgenic rat model of SOD 1 mutant-mediated amyotrophic lateral sclerosis (ALS). Proc Natl Acid Sci USA 2002; 99: 1604-1609].

The present work was undertaken to evaluate the effect of masitinib on Amyotrophic Lateral Sclerosis in SOD1 G93A rats. The effect of masitinib (40 mg/kg) was evaluated using SOD1G93A rats. These rats develop a motor syndrome with symptoms and pathological features of the human disease. Animals were observed weekly for onset of disease symptoms, as well as progression to death during 12 weeks of treatment.

Compared to control, masitinib prolonged the survival of SOD1G93A rats, without showing paralytic symptoms or weight loss. These results show a protective effect of masitinib in Amyotrophic Lateral Sclerosis.

1.1. Materials and Methods

1.1.1. Masitinib

Storage conditions: +4° C. as powder protected from light.

1.1.2 Drug Preparation

Masitinib was dissolved in water at a dose of 40 mg/kg.

1.2. Test Animals

Male hemizygous NTac:SD-TgN(SOD1 G93A)L26H rats (Taconic, US), originally developed by Howland, et al., were bred locally as outbred Sprague-Dawley background.

1.3. Treatment Schedule

SOD1G93A males rats aged 120 days were treated with 40 mg/kg of masitinib or placebo (vehicle), 5 times a week (Monday to Friday) by oral administration. At week 6, masitinib administration was switched to three times per week (40 mg/kg Monday, Wednesday and Friday).

1.4. Experimental Procedure

The response of masitinib in ALS was tested using a transgenic rat model of SOD1 mutant-mediated amyotrophic lateral sclerosis. Male SOD1G93A rats were divided randomly into the masitinib and vehicle groups (n=6 per group). Masitinib was freshly prepared in 500 μL of water (final volume) and administrated once a day from Monday to Friday. However, two rats from the masitinib group developed early signs of paralysis after and died before week 6. In order to prevent potential masked adverse effects, at week 6 the administration schedule was amended to three times a week (Monday, Wednesday and Friday) at the same dose.

Treatment was started while all rats were asymptomatic and having comparable stable weight (average 340 g/rat). Animals were observed weekly for onset of disease symptoms, as well as progression to death. Onset of disease was scored as the first observation of abnormal gait or overt hind limb weakness. End-stage of the disease was scored as complete paralysis of both hind limbs and the inability of the animals to right themselves after being placed on their side.

1.5. Results

As shown by the Kaplan-Meier plot of survival analysis (FIG. 1), after week 6 rats in the masitinib group appeared to develop the disease later and survived longer than the rats treated with vehicle only. Two rats from the masitinib group have survived 7 months without showing paralytic symptoms or weight loss. Hence, this animal model of ALS showed some degree of therapeutic benefit, providing a proof-of-principle that c-KIT inhibitors may ameliorate neurodegenerative diseases such as ALS.

### Example 2

### Effect of Masitinib on Amyotrophic Lateral Sclerosis in SOD1G93A Mouse Model

This work was undertaken to evaluate the effect of masitinib on Amyotrophic Lateral Sclerosis using a SOD1G93A mouse model.

The efficacy of masitinib was assessed in terms of time to disease onset and survival. Masitinib was administered at 30 or 100 mg/kg/day by oral gavage from Monday through Friday. Treatment started at 90 days-old animals before symptoms onset and continued until death. Assessments including weight, grip strength and righting reflex were performed at 12 weeks (baseline) and weekly until animals were unable to right themselves (18-20 weeks).

Administration of masitinib by oral gavage to B6SJL-Tg SOD1G93A female mice significantly retarded time to disease onset, as evidenced by a delay in time to symptoms onset, improved grip strength, and improved weight loss as compared with control B6SJL-Tg SOD1G93A animals. Moreover, the treatment with masitinib showed a trend to improve survival as evidenced by the righting reflex test. These results are encouraging considering that there is no treatment available to Amyotrophic Lateral Sclerosis other than riluzole, which delays death by only few months in humans.

2.1. Drug Preparation and Treatment Schedule

Masitinib was administered at 30 or 100 mg/kg/day concentration diluted in water by oral gavage from Monday through Friday.

2.2. Animals

Groups of B6SJL-Tg SOD1G93A female mice were treated as indicated above. Specifically, 8 B6SJL-Tg SOD1G93A mice received masitinib at 30 mg/kg/day; 8 SOD1G93A mice received masitinib at 100 mg/kg/day; 8 B6SJL-Tg SOD1G93A mice received water (i.e. vehicle, positive control); 4 Non-Tg SOD1G93A mice received water (i.e. negative control); and 4 Non-Tg SOD1G93A mice received masitinib at 100 mg/kg/day (i.e. negative intervention control). Treatment started at 90 days-old animals before symptoms onset and until death. Assessments including weight, grip strength and righting reflex were performed at 12 weeks (baseline) and weekly until animals were unable to right themselves (18-20 weeks).

| Experimental groups | SOD1^{G93A} | Non Tg |
|---|---|---|
| control | 8 female | 4 female |
| Masitinib 30 mg/Kg/day | 8 female | |
| Masitinib100 mg/kg/day | 8 female | 4 female |

Body weight was measured twice weekly from week 12 until completion of the study. Grip strength was measured weekly beginning at week 12 (baseline measure). Daily righting reflex was commenced at week 16 and animals that failed to right themselves were considered moribund and euthanized.

2.3. Experimental Procedure

Behavior and Assessment: Animals were observed every morning for onset of disease symptoms, as well as progression to death. Onset of motor neuron disease was scored as the first observation of an abnormal gait or evidence of hind-limb weakness.

End-stage of disease was scored as complete paralysis of both hind-limbs and the inability of the animals to right after being turned on a side.

Grip-strength test: The grip strength was obtained by pulling the animal backwards along the platform until the animal's hind paws grab the mesh grip piece on the push-pull gauge (San Diego Instruments, San Diego, CA). The animal was gently pulled backwards with consistent force by the experimenter until it released its grip. The hind limb grip force was recorded on the strain gauge. The resulting number was the average from 5 measurements in the same animal. After testing animals were placed back into the home cage. Righting reflex test: The righting reflex was used as a surrogate for survival. The animal is simply placed on its back on a flat surface and the time taken to right itself was measured (up to 0.5 min). Only one trial per mouse at each time point was performed.

2.4. Results

1) Onset and survival in masitinib treated B6SJL-Tg SOD1G93A mice.

The following table summarizes the results of masitinib administration (30 or 100 mg/kg/day from Mondays to Fridays) in B6SJL-Tg SOD1G93A mice on the age of symptoms onset and survival. There was a significant delay of symptoms onset with both masitinib doses.

| | | | | | | | | | | Age of | Age of |
|---|---|---|---|---|---|---|---|---|---|---|---|
| mouse # | treatment | Age of onse [symbol] | Age of deal [symbol] | mouse # | treatment | Age of onse [symbol] | Age of deal [symbol] | mouse # | treatment | onse [symbol] | deal [symbol] |
| VII88 | control | 98 | 125 | VII89 | Masi 30 mg | 104 | 150 | VII99 | Masi 100 m [symbol] | 111 | 135 |
| I1 | control | 98 | 127 | VII98 | Masi 30 mg | 98 | 124 | I7 | Masi 100 m [symbol] | 111 | 136 |
| I3 | control | 98 | 121 | I4 | Masi 30 mg | 111 | 128 | I15 | Masi 100 m [symbol] | 111 | 132 |
| I6 | control | 98 | 129 | I13 | Masi 30 mg | 119 | 147 | I33 | Masi 100 m [symbol] | 115 | 135 |
| I16 | control | 115 | 150 | I29 | Masi 30 mg | 111 | 139 | I39 | Masi 100 m [symbol] | 111 | 132 |
| I36 | control | 108 | 134 | I37 | Masi 30 mg | 110 | 147 | I45 | Masi 100 m [symbol] | 109 | 125 |
| I46 | control | 104 | 144 | I44 | Masi 30 mg | 119 | 141 | I48 | Masi 100 m [symbol] | 111 | 132 |
| I49 | control | 91 | 150 | I47 | Masi 30 mg | 119 | 141 | I43 | Masi 100 m [symbol] | 109 | 130 |
| Averages | | 101.25 | 135 | | | 111.375 | 139.625 | | | 111 | 132.125 |

[symbol]

indicates data missing or illegible when filed

FIG. 2 shows Kaplan-Meyer symptoms onset (left -2A) and survival (right -2B) curves from B6SJL-Tg SOD1 G93A mice treated with masitinib at 30 mg/kg/day (Masi 30), masitinib at 100 (Masi 100) mg/kg/day, or water (Ctrl) as indicated. Masitinib delayed the time to symptoms onset with respect to the positive control group at both concentrations used. Negative control animals, i.e. Non-Tg animals treated with masitinib at 100 mg/kg/day or water showed similar results to one another.

The Gehan-Breslow statistic for the onset curves was greater than would be expected by chance; there was a statistically significant difference between onset curves (P=0.002). To isolate the group or groups that differ from the others a multiple comparison procedure was performed. Both B6SJL-Tg SOD1G93A masitinib groups generated a treatment benefit that was significantly different from the positive control group, whereas no statistical difference was observed between the two masitinib doses.

All Pairwise Multiple Comparison Procedures (Holm-Sidak method):

Overall significance level=0.05

| | | Unadjusted | Critical | |
|---|---|---|---|---|
| Comparisons | Statistic | P Value | Level | Significant? |
| CTRL vs. MASI 2 | 7.837 | 0.00512 | 0.0170 | Yes |
| CTRL vs. MASI 1 | 6.892 | 0.00866 | 0.0253 | Yes |
| MASI 1 vs. MASI 2 | 0.781 | 0.377 | 0.0500 | No |

CRTL is Tg-control group; MASI 1 is masitinib at 30 mg/kg/day treatment group; MASI 2 masitinib at 100 mg/kg/day treatment group.

Additionally, treatment with masitinib showed a trend to improve survival. The median survival of B6SJL-Tg SOD1G93A mice treated with masitinib at 30 mg/kg/day (Masi 30) was higher compared with the positive control group (Ctrl) group (FIG. 2A).

2) Grip strength records from masitinib treated SODG93A mice.

FIG. 3 shows hind-limb grip strength for B6SJL-Tg SODG93A mice treated with masitinib at 30 or 100 mg/kg/day, B6SJL-Tg SODG93A mice treated with water (Tg Ctrl, positive control), and Non-Tg SODG93A mice (Non-Tg Ctrl, negative control). Because the masitinib treated and water treated Non-Tg animals exhibited no discernible difference in results these groups were pooled into a single Non-Tg control group (n=8). Masitinib at 30 mg/kg/day (Tg Masi 30 mg) significantly improved the grip strength curve as compared with the control (Tg Ctrl) group. Masitinib at 100 mg/kg/day (Tg Masi 100 mg) improved the grip strength during the first stage of the disease (from 90 to 120 days) as compared with the control (Tg Ctrl) group. Each data point is the mean±SEM from 8 animals per group.

Statistics analysis: The One Way Repeated Measures Analysis of Variance among treatment groups was greater than would be expected by chance; i.e. there was a statistically significant difference (P=<0.001) between the masitinib treatment groups and negative control group with respect to the positive control group.

3) Weight records from masitinib treated SODG93A mice.

FIG. 4 shows weight records for B6SJL-Tg SODG93A mice treated with masitinib at 30 or 100 mg/kg/day, B6SJL-Tg SODG93A mice treated with water (Tg Ctrl, positive control), and Non-Tg SODG93A mice (Non-Tg Cntl, negative control). Because the masitinib treated and water treated Non-Tg animals exhibited no discernible difference in results these groups were pooled into a single Non-Tg control group (n=8). Masitinib at 30 mg/kg/day (Tg Masi 30 mg) reduced weight loss during the first stage of the disease (from 90 to 120 days) as compared with the control (Tg Ctrl) group. Each data point is the mean±SEM from 8 animals per group.

2.4. Conclusions

Administration of masitinib by oral gavage to B6SJL-Tg SOD1G93A female mice significantly retarded time to disease onset, as evidenced by a delay in time to symptoms onset, improved grip strength, and improved weight loss as compared with control B6SJL-Tg SOD1G93A animals. Moreover, the treatment with masitinib showed a trend to improve survival as evidenced by the righting reflex test. These results are encouraging considering that there is no treatment available to ALS other than riluzole, which delays death by only few months in humans.

Taken together these data (examples 1 and 2) indicate that masitinib might offer therapeutic benefits in ALS patients. These data justify commencement of a full human clinical trial to confirm the observed treatment effect of masitinib in ALS and characterize the optimal treatment procedure (i.e. patient subpopulations of special interest and dosing regimens).

### Example 3

### Clinical Study Protocol

Study design: Multicenter, randomized, double-blind, placebo-controlled, parallel group, phase 2/3 study to compare the efficacy and safety of masitinib in combination with riluzole versus placebo in combination with riluzole in the treatment of patients suffering from Amyotrophic Lateral Sclerosis (ALS)
Diagnosis: Patients with definite or probable ALS.
Study treatment: Masitinib 100 and 200 mg tablets.
Associated product: Placebo, matching 100 mg and 200 mg tablets.
Duration of treatment: 48 weeks of study treatment with possible extension.

The objective is to compare the efficacy and safety of masitinib combined with riluzole versus placebo combined with riluzole in the treatment of patients suffering from ALS. Eligible patients will be treated during 48 weeks and patients will be proposed to enter a double-blind extension phase. As soon as the treatment groups will be known, patients receiving placebo will be withdrawn from the study. Patients treated with masitinib will be allowed to continue their treatment at the same dose level providing that the benefit/risk balance is still in favor of masitinib continuation according to the investigator resulting in an absence of progression and a good tolerance.

Study treatment will be discontinued in case of:
Informed consent withdrawal
Adverse or undercurrent event considered intolerable by the patient or incompatible with continuation of the study according to the investigator
Protocol violation (e.g., noncompliance with treatment administration, prohibited treatment needed)

Patients enrolled will be randomized in 3 groups:
Group 1: 70 patients will receive masitinib 4.5 mg/kg/day and riluzole
Group 2: 70 patients will receive masitinib at 3 mg/kg/day and riluzole
Group 3: 70 patients will receive placebo and riluzole

Subjects enrolled will receive a total daily dose of 4.5 or 3 mg/kg masitinib, or a matching placebo, to be taken during meals as indicated in the Tables 1 and 2 below:

Masitinib is supplied as 100 mg and 200 mg tablets (respectively corresponding to 119.3 mg and 238.5 mg of the mesylate salt of masitinib) packaged in polyethylene bottles closed with a childproof cap. Inactive ingredients are microcrystalline cellulose, povidone, crospovidone, magnesium stearate and coating agent, Opadry orange.

Placebo is supplied in tablets identical to the masitinib ones, with the same composition except for active ingredient.

All medications supplied by the Sponsor to be used in this study will have been manufactured, tested, and released according to current GMP guidelines.

Study treatment daily dose of 4.5 mg/kg will be administered in divided doses as indicated in Table 1.

TABLE 1
Dose of study treatment (mg) to be administered according to patient's
weight (4.5 mg/kg/day)

| | | 4.5 mg/kg/day | | |
|---|---|---|---|---|
| | | Daily dose | | |
| Patient's weight in kg | | (mg) | Morning* (mg) | Evening** (mg) |
| | ≦55.5 | 200 | 100 | 100 |
| >55.5 | 77.7 | 300 | 100 | 200 |
| >77.7 | 99.9 | 400 | 200 | 200 |
| >99.9 | | 500 | 200 | 200 + 100 |

*Morning: the tablets should be taken during breakfast. In case of nausea, the administration can take place during lunch.
**Evening: the tablets should be taken during dinner.

Morning: the tablets should be taken during breakfast. In case of nausea, the administration can take place during lunch. **Evening: the tablets should be taken during dinner.

Study treatment daily dose of 3 mg/kg will be administered in divided doses as indicated in Table 2.

TABLE 2
Dose of study treatment (mg) to be administered according to patient's
weight (3 mg/kg/day)

| | | 3 mg/kg/day | | |
|---|---|---|---|---|
| | | Daily dose | | |
| Patient's weight in kg | | (mg) | Morning* (mg) | Evening** (mg) |
| | ≦49.9 | NOT POSSIBLE | | |
| >49.9 | 83.3 | 200 | 100 | 100 |
| >83.3 | | 300 | 100 | 200 |

*Morning: the tablets should be taken during breakfast. In case of nausea, the administration can take place during lunch.
**Evening: the tablets should be taken during dinner.

According to the initial dose 3 mg/kg/day of masitinib or matching placebo, the steps for the dose reduction are as follows:

| Starting dose | 1^{st} dose reduction | 2^{nd} dose reduction |
|---|---|---|
| 3 mg/kg/day | 1.5 mg/kg/day | STOP |

TABLE 3
Dose of study treatment to be administered according to patient's weight,
after a dose reduction to 1.5 mg/kg/day (randomization dose: 3 mg/kg/day)

| | | 1.5 mg/kg/day | | |
|---|---|---|---|---|
| | | Daily dose | | |
| Patient's weight in kg | | (mg) | Morning* (mg) | Evening** (mg) |
| | ≦49.9 | Stop | | |
| >49.9 | 83.3 | 100 | | 100 |
| >83.3 | | 200 | 100 | 100 |

*Morning: the tablets should be taken during breakfast. In case of nausea, the administration can take place during lunch.
**Evening: the tablets should be taken during dinner.

According to the initial dose 4.5 mg/kg/day of masitinib or matching placebo, the steps for the dose reduction are as follows:

| Starting dose | 1^{st} dose reduction | 2^{nd} dose reduction | 3^{rd} dose reduction |
|---|---|---|---|
| 4.5 mg/kg/day | 3 mg/kg/day | 1.5 mg/kg/day | STOP |

TABLE 4
Dose of study treatment to be administered according to patient's weight,
after a dose reduction to 3 mg/kg/day (randomization dose: 4.5 mg/kg/day)

| | | 3 mg/kg/day | | |
|---|---|---|---|---|
| | | Daily dose | | |
| Patient's weight in kg | | (mg) | Morning* (mg) | Evening** (mg) |
| | ≦55.5 | | STOP | |
| >55.5 | 83.3 | 200 | 100 | 100 |
| >83.3 | | 300 | 100 | 200 |

*Morning: the tablets should be taken during breakfast. In case of nausea, the administration can take place during lunch.
**Evening: the tablets should be taken during dinner.

TABLE 5
Dose of study treatment to be administered according to patient's weight,
after a dose reduction to 1.5 mg/kg/day
(randomization dose: 4.5 mg/kg/day)

| | | 1.5 mg/kg/day | | |
|---|---|---|---|---|
| | | Daily dose | | |
| Patient's weight in kg | | (mg) | Morning* (mg) | Evening** (mg) |
| | ≦55.5 | | STOP | |
| >55.5 | 99.9 | 100 | | 100 |
| >99.9 | | 200 | 100 | 100 |

*Morning: the tablets should be taken during breakfast. In case of nausea, the administration can take place during lunch.
**Evening: the tablets should be taken during dinner.

No dose escalation will be authorized for patients who have had a dose reduction for safety reasons. Procedure in case of missed or vomited doses of study treatment tablets:
In case the morning dose has been missed, it can be taken until 2 pm. on the same day. Should it be later than 2 pm, the missed dose will not be made up and study treatment will be resumed at the evening dose on the same day.
In case the evening dose is missed, it should not be made up the day after in addition to the morning dose. The study treatment will be resumed the day after as scheduled in the protocol.
Should the patient vomit within 10 minutes after the last study treatment dose intake, another dose should be taken.

Should the patient vomit later than 10 minutes following the last study treatment dose intake, study treatment will be resumed at the next theoretical dose intake, but the last dose will not be replaced.

1-19. (canceled)

20. A method for treating amyotrophic lateral sclerosis (ALS) in a mammal in need thereof, wherein said method comprises administering to the mammal at least one tyrosine kinase inhibitor, mast cell inhibitor or c-Kit inhibitor.

21. The method of claim 20, wherein said mammal is a human patient.

22. The method of claim 20, wherein said tyrosine kinase inhibitor or mast cell inhibitor is an inhibitor of kinase activity selected from the tyrosine kinases of: c-Kit, PDGFR, Lyn and Fyn.

23. The method of claim 20, wherein said tyrosine kinase inhibitor, mast cell inhibitor or c-Kit inhibitor is masitinib or a pharmaceutically acceptable salt thereof.

24. The method of claim 20, wherein said tyrosine kinase inhibitor, mast cell inhibitor or c-Kit inhibitor is a mesilate salt of masitinib.

25. The method of claim 20, wherein said tyrosine kinase inhibitor, mast cell inhibitor or c-Kit inhibitor is administered at a daily dose of 1.0 to 12.0 mg/kg/day (mg per kg bodyweight per day).

26. The method of claim 20, wherein said tyrosine kinase inhibitor, mast cell inhibitor or c-Kit inhibitor is administered at a dose of 2.5 to 9.5 mg/kg/day.

27. The method of claim 20, wherein said tyrosine kinase inhibitor, mast cell inhibitor or c-Kit inhibitor is administered at a dose of 3.0 to 9.0 mg/kg/day.

28. The method of claim 20, wherein said tyrosine kinase inhibitor, mast cell inhibitor or c-Kit inhibitor is administered at a dose of 1.5, 3.0, 4.5, 6.0, 7.5, or 9.0 mg/kg/day.

29. The method of claim 20, wherein said tyrosine kinase inhibitor, mast cell inhibitor or c-Kit inhibitor is administered orally.

30. The method of claim 20, wherein said tyrosine kinase inhibitor, mast cell inhibitor or c-Kit inhibitor is administered once or twice a day.

31. The method of claim 20, wherein said tyrosine kinase inhibitor, mast cell inhibitor or c-Kit inhibitor is administered in combination with at least one other pharmaceutically active ingredient.

32. The method of claim 20, wherein said tyrosine kinase inhibitor, mast cell inhibitor or c-Kit inhibitor is administered in combination with an antiglutamate compound and/or an inhibitor of glutamate carboxypeptidase II.

33. The method of claim 20, wherein said tyrosine kinase inhibitor, mast cell inhibitor or c-Kit inhibitor is administered in combination with an antiglutamate compound selected from the group comprising riluzole (6-(trifluoromethoxy)benzothiazol-2-amine), topiramate (2,3:4,5-Bis-O-(1-methylethylidene)-beta-D-fructopyranose sulfamate), gabapentin (2-[1-(aminomethyl)cyclohexyl]acetic acid), lamotrigine (6-(2,3-dichlorophenyl)-1,2,4-triazine-3,5-diamine), talampanel ((8R)-7-Acetyl-5-(4-aminophenyl)-8,9-dihydro-8-methyl-7H-1,3-dioxolo[4,5-h][2,3]benzodiazepine), and ceftriaxone ((6R,7R)-7-[[(2Z)-2-(2-amino-1,3-thiazol-4-yl)-2-methoxyiminoacetyl]amino]-3-[(2-methyl-5,6-dioxo-1H-1,2,4-triazin-3-yl)sulfanylmethyl]-8-oxo-5-thia-1-azabicyclo[4.2.0]oct-2-ene-2-carboxylic acid); and/or an inhibitor of glutamate carboxypeptidase II.

34. The method of claim 20, wherein said tyrosine kinase inhibitor, mast cell inhibitor or c-Kit inhibitor is administered in combination with riluzole.

35. The method of claim 20, wherein said tyrosine kinase inhibitor, mast cell inhibitor or c-Kit inhibitor is administered in combination with at least one pharmaceutically active ingredient in a combined preparation for simultaneous, separate, or sequential use.

36. A pharmaceutical composition or kit comprising a tyrosine kinase inhibitor, mast cell inhibitor or c-Kit inhibitor and an antiglutamate compound and/or an inhibitor of glutamate carboxypeptidase II.

37. The pharmaceutical composition or kit of claim 36, wherein the antiglutamate compound is selected from the group comprising riluzole, topiramate, gabapentin, lamotrigine, talampanel, and ceftriaxone.

38. The pharmaceutical composition or kit of claim 36, wherein said tyrosine kinase inhibitor, mast cell inhibitor or c-Kit inhibitor is masitinib and wherein said antiglutamate compound is riluzole.
